(19) United States
(12) Patent Application Publication
(10) Pub. No.: US 2026/0240613 A1
(43) Pub. Date: Aug. 20, 2026

(54) AUTOMATED AEROENGINE BLADE DETECTION, WEIGHING AND SORTING

(71) Applicants: Khalifa University of Science and Technology, Abu Dhabi (AE); Aerospace Holding Company LLC, Abu Dhabi (AE)

(72) Inventors: Mohammed Ramadan, Quebec (CA); Laith AbuAssi, Abu Dhabi (AE); Abdulla Hasan Ayyad, Abu Dhabi (AE); Abdelrahman Youssef Elmaghawry Sayed Ahmed, Abu Dhabi (AE); Yusra Abdulrahman, Abu Dhabi (AE); Yahya Zweiri, Abu Dhabi (AE); Brain Moyo, Abu Dhabi (AE); Aiswarya Babu, Abu Dhabi (AE); Abdallah Mohammad Alkilany, Abu Dhabi (AE)

(21) Appl. No.: 19/544,181

(22) Filed: Feb. 19, 2026

Related U.S. Application Data
(60) Provisional application No. 63/760,235, filed on Feb. 19, 2025.

(51) Int. Cl. A61B 34/30; B25J 9/16; B25J 19/02

(52) U.S. Cl. CPC A61B 34/30; B25J 9/1664; B25J 9/1679; B25J 9/1697; B25J 19/023

(57) ABSTRACT

A system may leverage an artificial intelligence (AI) to support a robotic system and camera system associated with the robotic system for weighing aerospace engine blades. The system may autonomously gather serial and mass data from aerospace engine blades in a tray, manipulate a robotic arm to grasp the aerospace engine blade and gain a mass measurement of the aerospace engine blade. The robotic system may then sort the blades by descending mass for installation in an aerospace engine.

### CROSS-REFERENCES TO RELATED APPLICATIONS

This application claims benefit to U.S. Provisional Patent Application No. 63/760,235, filed Feb. 19, 2025, the entire contents of which are hereby incorporated in their entirety for all purposes.

### BACKGROUND

The rise of smart technology and automation has dramatically reshaped the manufacturing landscape, with industrial automation leading this transformation and involving robotic manipulators in industries such as aerospace and automotive. The availability of affordable computational power and the widespread adoption of industrial robotic arms have enabled automation in tasks such as robotic machining, precise manipulation, and automated inspections. The aerospace industry constantly evolves and has strict maintenance requirements. As such, some challenges can include the implementation of automation within maintenance, repair, and overhaul (MRO) operations due to the complexity of aerospace components, to integration with existing processes, and the desire for high reliability and safety standards. It can be difficult to integrate inspections of aircraft components, such as wings and fastener holes, with advanced computer vision and image processing techniques.

### BRIEF SUMMARY OF THE INVENTION

The present disclosure involves a sophisticated vision-guided robotic system designed for the automated detection, weighing and sorting of aero-engine blades. The system can be engineered to address the inefficiencies and errors inherent in other processes. The system can include a specialized end effector that integrates a high-precision load cell and an imaging sensor, facilitating accurate and rapid blade weighing coupled with advanced robotic perception capabilities.

In some examples, a system can include a robotic manipulator, an end effector, a measurement unit, an imaging sensor, and a computing system The end effector can include a gripping mechanism coupled with the robotic manipulator to facilitate manipulation of one or more aerospace engine blades of an aerospace engine. The measurement unit can be coupled with the robotic manipulator to perform mass and weight measurements of the one or more aerospace engine blades as the one or more aerospace engine blades are gripped by the robotic manipulator. The imaging sensor can be communicatively coupled with the robotic manipulator. The end effector, and/or an imaging system can be used for capturing image data about the one or more aerospace engine blades. The computing system can be configured to perform various operations. The operations can include detecting the image data about the one or more aerospace engine blades using a neural network. The operations can include refining the detected image data for detecting the one or more aerospace engine blades. The operations can include causing the robotic manipulator, using the end effector, to grasp and measure an aerospace engine blade of the one or more aerospace engine blades such that a mass measurement of the aerospace engine blade is generated by the measurement unit. The operations can include sorting mass of the aerospace engine blade using the neural network by comparing the mass measurement to masses of other aerospace engine blades of the one or more aerospace engine blades being measured. The operations can include sorting, based on output from the neural network, the one or more aerospace engine blades based on the mass measurement and according to a dynamic balancing setting associated with the aerospace engine. The operations can include outputting a command comprising instructions executable to display the data throughout a process, including an optimized installation slot in the aerospace engine for each of the aerospace engine blades to ensure dynamic balancing.

### BRIEF DESCRIPTION OF THE DRAWINGS

Various embodiments in accordance with the present disclosure will be described with the present disclosure will be described with reference to the drawings in which in accordance with at least one embodiment.

FIG. 1 is a schematic view of an example of an autonomous robotic system that may be utilized to measure and sort an object in accordance with at least one embodiment.

FIG. 2 is a schematic of an example of the main components of an autonomous robotic system in accordance with at least one embodiment.

FIG. 3 is a data flow diagram of an example of a process of an autonomous robotics system measuring the mass of the blades within a tray in accordance with at least one embodiment.

FIG. 4 shows examples of a robotic system sorting sequential blocks of aerospace engine blades in accordance with at least one embodiment.

FIG. 5 is a schematic of an autonomous robotic system with a camera system and end effector in accordance with at least one embodiment.

FIG. 6 is a chart of an example of blade measurements that may be acquired from an autonomous robotic systemin accordance with at least one embodiment.

FIG. 7 is a data flow diagram of an example of a process of installing aerospace engine blades into rotors after being sorted in descending mass order in accordance with at least one embodiment.

FIG. 8 is a chart of an example of an output of the sorting system of the autonomous robotic system in accordance with at least one embodiment.

FIG. 9 is a flowchart of an example of process for an autonomous robotic system measuring and sorting aerospace engine blades in accordance with at least one embodiment.

FIG. 10 is a block diagram of an example of a computer system that can be used to control a robotic manipulator with an inspection system in accordance with at least one embodiment.

### DETAILED DESCRIPTION OF THE INVENTION

Certain aspects and features of the present disclosure relate to an autonomous robotic system that may employ an artificial intelligence (AI) model for the autonomous robotic system to detect weight and to sort aerospace engine blades. The AI model may be or include a neural network system that may be employed for blade detection and localization refinement. The autonomous robotic system may utilize multiple sensors, manipulate an object, and generate a result to an operator or system. The robotic system may be attached to a command computer. The AI model may utilize a camera system within the robotic system to localize the blades and blade tray. The AI model of the robotic system can adapt the manipulation of the joints of the autonomous robotic system, and the AI model can increase efficiency and precision as it may relate to the weight and sorting of the aerospace engine blades.

A camera system may be embedded within the autonomous robotic system to minimize calibration and may be installed in an eye-in-hand configuration on the autonomous robotic system. In some embodiments the camera system may include an Intel RealSense D405 camera, though other camera systems are possible as substitutes. A devised detector network, such as YOLOv5, may be used to store an aerospace engine blade in a tray and store the pixel coordinates of the stored aerospace engine blade as coordinates that may be represented as, for example, [u, v]. The devised detector network can use an AI model, such as a neural network, to identify the blades and location of the blades within a tray. The blades can also be tracked by the devised detector network and AI using the mass and serial numbers to support sorting functions. Pixel coordinates may be normalized to a range, for example between 0.0 and 1.0, and a bounding box may be developed internally to the devised detector network centering around the coordinate range. A bounding box may include a rectangular area that encloses a specific region of interest and may include the use of computer vision. The pixel coordinates may be transformed into real world coordinates relative to the autonomous robotic system and may allow for the blades to be associated with a slot of a tray through a Unified Robotic Description Format (URDF). A URDF description may include XML-based markup language to define geometry of the autonomous robotic system and may allow for the autonomous robotic system to identify a minimum Euclidean distance to the blade from the slot.

The autonomous robotic system may mimic manual robotic systems that may be present in industrial settings. The autonomous robotic system may also include a robot with joints and an end effector that may be manipulated to precisely imitate grasping poses and grasp objects. The end effector may include an EGP 40-N-N-B Schunk gripper, and in some embodiments the end effector may include or be a pneumatic gripper. The autonomous robotic system may also employ an S-beam load cell mass measurement tool, and in some embodiments the mass measurement tool may include a DBSMM-2 KG-002 module, which can be in line with the end effector. An S-beam load cell mass measurement tool may be a specialized load distribution too which tracks deformations of a metal beam inside the tool as a weight is applied, which may allow for precise weight measurements.

The camera system may be employed to localize the target blade. The camera system may record the bounding box of the blade and may use fiducial markers or serial data to locate the blade. The autonomous robotic system may position its joints near the aerospace engine blade and record an initial reading Mᵢₙᵢₜᵢₐₗ of the weight of a load free measurement. The robotic system may grasp the blade and record a M^{j} measurement of the load cell measuring the aerospace engine blade once the blade is aligned with the grippers Z-axis. The measurements may be passed through a low pass filter to average multiple measurements as M_{final}=ΣⱼM^{J}_{final}/N, j∈[1, N] in which N may represent the number of measurements. An M_{blade}=M_{final}−Mᵢₙᵢₜᵢₐₗ calculation may be completed by the autonomous robotic system and may be used to estimate the mass of the aerospace engine blade. The estimated weight can be reported to a graphical user interface (GUI) in real time, and the process is repeated N times across a set of dedicated aerospace engine blades within the tray. The blades may be sorted in descending order according to their mass into groups such as S1 and S2. S1 may include blades with the highest and lowest masses and S2 may contain the second highest and lowest masses. Aerospace engine blades may be sequentially added to S1 and S2. The third highest and third lowest masses may be added to S1, and this process may continue in a similar manner until the blades are sorted. Once sorted S1 may be placed on top of S2 resulting in a balanced sorting order adhering to the specified requirements of jet engine rotors.

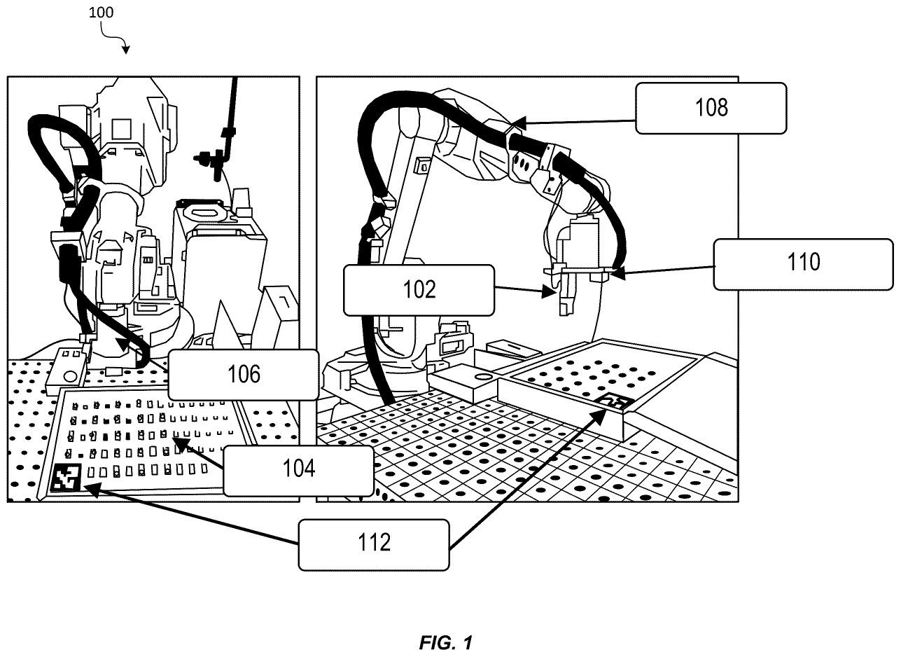

FIG. 1 is a schematic view of an example of an autonomous robotic system 100 that may be utilized to measure and sort an object. The proposed system, as illustrated in FIG. 1, may include an integration of machine vision and robotics. As the system can use an artificial intelligence (AI) model for the detection and localization of aero-engine blades 104, the system's end effector 106 can be equipped with a gripper 102 capable of securely handling the aero-engine blades 104. The mass of each blade of the aero-engine blades 104, or any subset thereof, can be measured with high precision as the robot picks and manipulates the aero-engine blades 104 using a load cell integrated into the gripper 102. In some embodiments, the measuring process is performed while the blade is being held or otherwise manipulated. Additionally, or alternatively, the system can include one or more processing units. The system software tracks the location and mass of each blade such as to ensure accurate sorting.

The system architecture can include several key components. For example, the key components can include a robotic arm 108 that may be included in the autonomous robotic system. The robotic arm 108 may include an industrial robot that maneuvers the end effector to the target grasping positions. In some embodiments an ABB 1600 10 is an example of a robotic arm that can be used.

Attached to the robotic arm 108 at one end may be an end effector 106. An example of the end effector 106 can include an EGP 40-N-N-B Schunk gripper 102 integrated with a high-precision load cell, for example a S-Beam Load Cell DBBSMM-2 kg-002 for blade mass measurement. The end effector 106 may be utilized to grasp and position certain objects including an aerospace engine blade. A camera system 110 may be attached to the end effector 106 in an eye-in-hand position for image capture by the autonomous robotic system 100. The camera system 110 may include an imaging sensor. An example of the imaging sensor can include an Intel RealSense D405 depth camera that can capture images of the blades and that can facilitate detection and localization of the blades.

The system can include a software system, such as a YOLOv5 artificial neural network, for blade detection and localization refinement. The software system can facilitate tracking and sorting the blades based on their mass and serial numbers such as to ensure precise and efficient sorting The software system may incorporate the camera system 110 to locate fiducial markers 112, which may be a code-type marker used in computer vision to identify important information for the AI model such as serial data and location.

The autonomous robotic system may include a processing unit. The processing unit may utilize an artificial intelligence (AI) system. The processing unit may also be attached to the computer control. The processing unit may be utilized to acquire measurements about the blades, process the measurements using one or more algorithms performing sorting logic, display data, and/or log the measured or processed data.

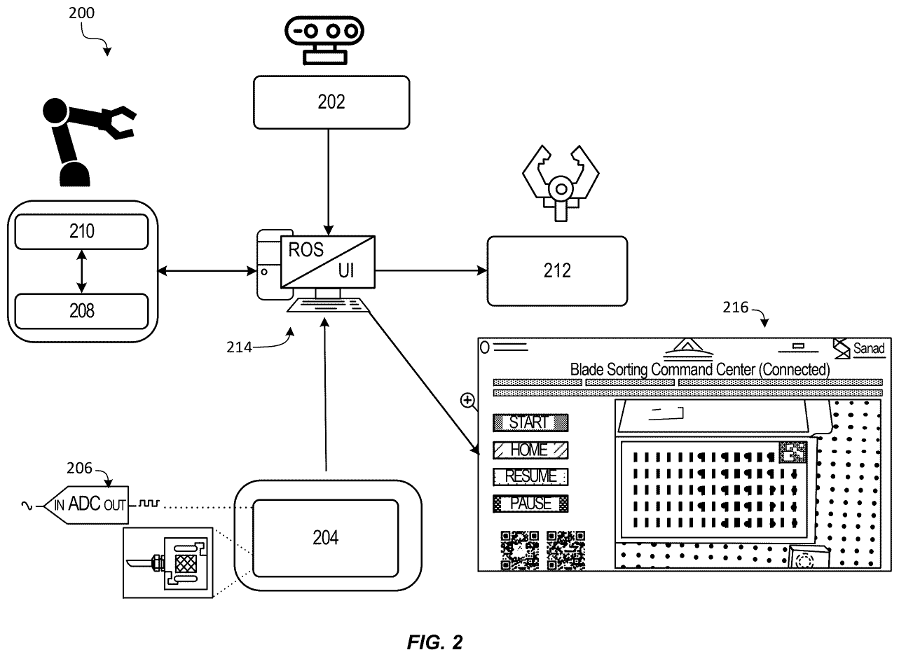

FIG. 2 is a schematic of an example of components of an autonomous robotic system. In some examples, FIG. 2 illustrates connections, as the command PC receives and processes measurements from different sensors, performs guidance and control logic for the manipulation of the robot, and generates the status and results to the operator using the designed graphical user interface (GUI). The computer and robotic system 200 can include two sensing modules, a depth camera 202 for visual feedback and guidance, and the S-beam load cell 204 for weight measurements. The S-beam load cell 204 can be connected to a digitizer 206 for analogue-to-digital (A/D) conversion, which may be linked to an RS232 to USB converter such as to establish a connection with a command computer. In some embodiments, the S-beam load cell 204 may be connected with a processing unit through signal processing units and/or data interface modules. The feedback from the depth camera 202 can be used to localize the blades and the blade tray within the robot workspace in three dimensions. The depth camera 202 can be used in an “eye-in-hand” configuration to minimize the effect of calibration errors, to enable a multi-stage blade localization process from different relative distances between the depth camera 202 and the blade, and/or to promote adaptation to different operation environments. Additionally, or alternatively, the depth camera 202 can be used in an “eye-to-hand” configuration or other suitable use configurations.

Using the blade's position, target trajectories for the robot manipulator can be forwarded to the robot controller, which can control the joints of the robot that may be controlled by a robot controller 208 linked to a robotic arm 210. The vision-guided robotic system for aero-engine blade weighing represents a significant advancement over traditional techniques. For example, the system offers increased efficiency, precision, and ease of integration over other techniques. The computer and robotic system may also be used to control a gripper 212 that may be attached to a robotic arm 210. The gripper 212 may be controlled independently of the robotic arm 210. A command computer 214 may display a graphical user interface (GUI) 216, displaying the functions of the computer and robotic system 200.

A devised detector network, such as YOLOv5s, can be used in which the detector network can detect the blades within the tray and store their pixel coordinates [u, v]. The coordinates can be transformed into world coordinates relative to the robot frame using the camera model and depth value estimate. Concurrently, the slots the blades can be positioned and can be located and associated with each blade by using a visual fiducial to localize the tray and the pre-know knowledge of the geometric dimensions of the slots within the tray. The association is based on finding the slot with the minimum Euclidean distance to the blade. A loop can be initiated to move to each blade and center it in the image plane. In order to detect the blades and localize their position in the image plane, a computer vision algorithm, such as Ultralytics YOLOv5s or newer, or other suitable computer vision algorithms, can be deployed, and the algorithm may detect objects at different scales and with high inference speed, which can allow for fast-paced automation of the process.

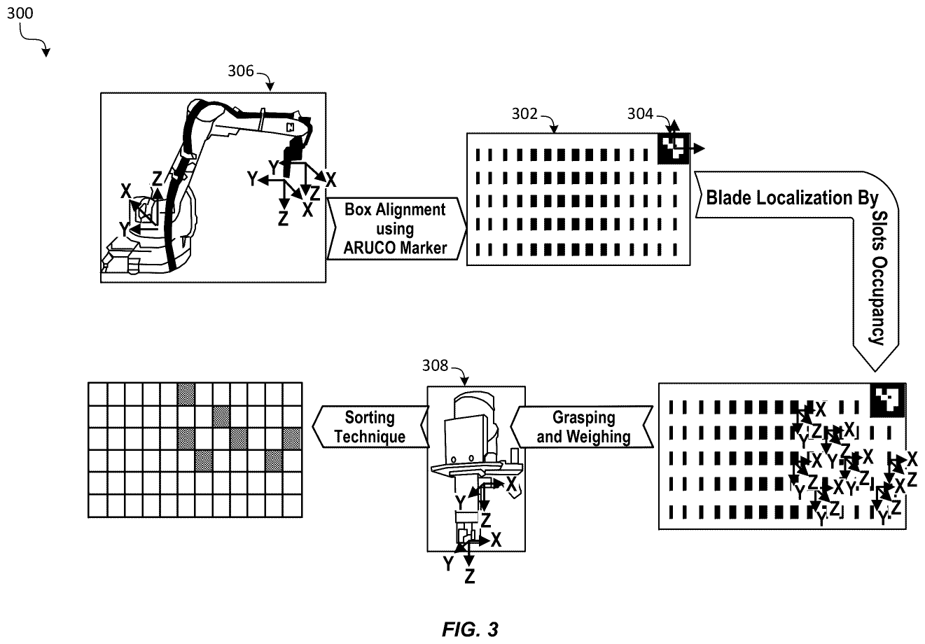

FIG. 3 is a data flow diagram of an example of a process of an autonomous robotics system measuring the mass of the blades within a tray 300. The robot can localize the tray 302 and the slots within it using a visual fiducial marker 304. The robot can localize the blades in the tray using a deep-learning model or similar computer vision algorithms. Subsequently, the detection of each blade is carried out in two stages. In the first stage, the system identifies each aero engine blade and determines its position relative to the robot's fixed frame 306. Once the blade's position is established, it can be assigned a corresponding slot number based on its location within the box. This ensures accurate association between each detected blade and its designated slot. After the blade association step, the second stage of blade localization is performed. The second stage can be the blade location refinement process. In this process, the robot moves the depth camera 202 of FIG. 2 to close proximity to each individual blade with a predefined standoff and re-performs the blade localization to refine the location estimate. Once the blade location estimate is refined the robot grips each blade and measures its mass using a load cell 308 embedded in the gripper 102 of FIG. 1. The processing unit can receive the mass measurements from the load cell and can process them using bias and drift offset along with low pass filtering. The system updates the GUI and reports the correct order to install the blades in the aero-engine.

The two-stage blade localization technique can ensure accurate detection of observed engine blades for achieving dynamic balancing of an engine. Additionally, or alternatively, the blade refinement technique can eliminate false detections during the first blade detection and localization stage, preventing outliers from being added to the balancing sequence when performing the blade operation on an aircraft engine. Moreover, the robustness in detecting and localizing blades can be used for precise grasping from desired end effector poses, impacting the precision of mass measurements during the grasping process. Ultimately, the system can have an advantage in its adaptability for deployment in production lines.

The performance of the system can be validated in terms of blade detection accuracy and localization of blade 3D positions. This can be done considering the proposed two-stage localization approach for blade detection and localization, which can be important as it addresses robustness against potential outliers generated by the devised detector. Given the robust detection and tracking of the blade 2D image points, the 2D points can be transformed into 3D positions, leveraging in-plane fiducial marker detection and accounting for their alignment with the fiducial marker. Table 1 and Table 2 present the repeatability, σₓ and σ_{y}, of the 3D blade localization in x and y dimensions, respectively. An example of the aggregate localization repeatability for the system is calculated to be approximately 0.265 mm. This validation process emphasizes the system's capability to consistently and accurately localize the 3D positions of the detected blades for precision weighing of the aeroengine blade.

TABLE 1
Position of the blades in X- axis

| Trial ID | 1 | 2 | 3 | 4 | 5 | 6 | 7 | 8 |
|---|---|---|---|---|---|---|---|---|
| 1 | −160.55 | −103.14 | −40.38 | −230.86 | −165.16 | −105.29 | −45.49 | 17.67 |
| 2 | −160.55 | −103.44 | −40.7 | −230.71 | −165.09 | −105.29 | −45.49 | 17.34 |
| 3 | −160.57 | −104.4 | −40.06 | −231.18 | −164.69 | −105.27 | −45.69 | 17.34 |
| 4 | −160.57 | −103.44 | −41.34 | −231.65 | −165.32 | −105.29 | −45.49 | 18.09 |
| 5 | −160.57 | −103.12 | −41.02 | −230.86 | −165.09 | −105.27 | −45.37 | 17.34 |
| 6 | −160.35 | −104.72 | −40.38 | −231.18 | −165.09 | −104.32 | −46.01 | 17.99 |
| 7 | −160.35 | −103.46 | −40.7 | −230.86 | −165 | −105.58 | −45.49 | 17.34 |
| 8 | −160.57 | −104.4 | −40.91 | −231.51 | −165.09 | −105.29 | −45.69 | 17.67 |
| σ | 0.1 | 0.64 | 0.41 | 0.34 | 0.18 | 0.37 | 0.2 | 0.31 |
| (mm) | | | | | | | | |
| | | | | | | | σₓ(mm) | 0.35 |

TABLE 2
Position of the blades in Y- axis

| Trial ID | 1 | 2 | 3 | 4 | 5 | 6 | 7 | 8 |
|---|---|---|---|---|---|---|---|---|
| 1 | −1164.2 | −1163.6 | −1160.4 | −1117.6 | −1112.4 | −1111 | −1111.2 | −1112.1 |
| 2 | −1163.9 | −1163.3 | −1159.5 | −1117.7 | −1112.5 | −1110.7 | −1111.2 | −1111.4 |
| 3 | −1163.9 | −1163.3 | −1160.1 | −1117.9 | −1112.7 | −1110.5 | −1111.1 | −1112.1 |
| 4 | −1163.9 | −1163.9 | −1158.2 | −1118 | −1112.3 | −1111 | −1111.2 | −1112.2 |
| 5 | −1164.2 | −1163.6 | −1160.7 | −1118.2 | −1112.2 | −1110.5 | −1111.1 | −1111.8 |
| 6 | −1164.2 | −1163.6 | −1157.9 | −1117.6 | −1112.5 | −1109.9 | −1111.1 | −1111.8 |
| 7 | −1164.2 | −1163.6 | −1157.9 | −1117.9 | −1112 | −1110.5 | −1110.9 | −1111.8 |
| 8 | −1164.2 | −1163.6 | −1157.5 | −1117.9 | −1112.5 | −1111 | −1110.8 | −1111.8 |
| σ | 0.16 | 0.2 | 1.3 | 0.22 | 0.21 | 0.36 | 0.16 | 0.25 |
| (mm) | | | | | | | | |
| | | | | | | | σ_{y}(mm) | 0.49 |

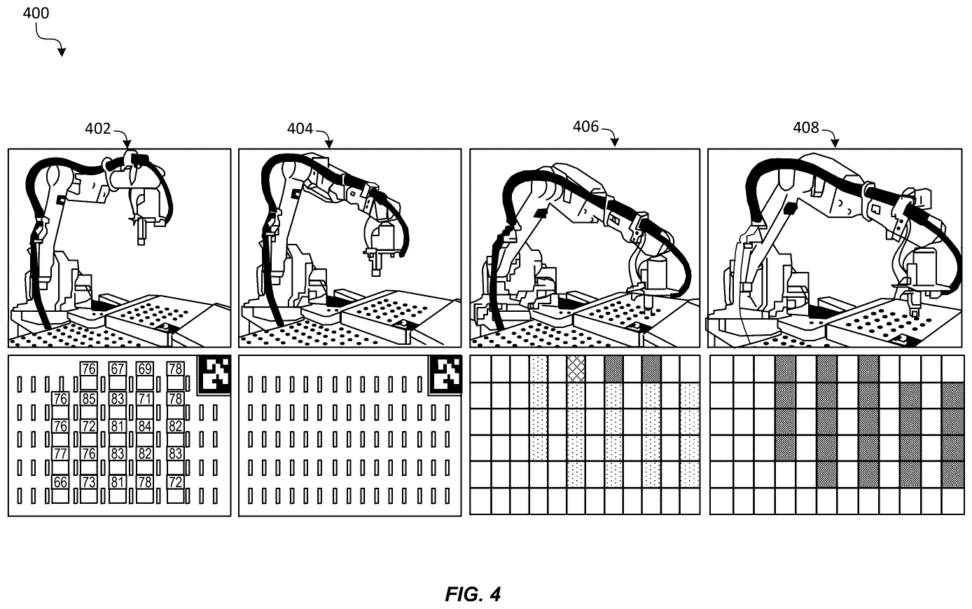

FIG. 4 illustrates examples of a robotic system sorting sequential blocks of aerospace engine blades 400. Part a) of FIG. 4 illustrates that the blades are initially detected using a devised detector as in 402. Part b) of FIG. 4 illustrates blade detection and localization refinement for blade localization robust against detection outliers as shown in 404. Part c) of FIG. 4 illustrates that following detection, the end effector moves towards the blade for grasping and undergoes precise weighing as shown in 406. Part d) of FIG. 4 illustrates that the process can be repeated for all blades until a sorting scheme is generated for dynamic balancing as shown in 408. That is, throughout various experimental trials, the system can follow a sequential process, as depicted in FIG. 4. The sorting scheme may start with an initial robot homing and blade detection sequence utilizing the artificial neural network. Once the blade is detected the location is refined and entered as a pixel coordinate. The robotic system may then proceed to grasp and weigh the aerospace engine blade. The aerospace engine blades may then be sorted for all blade mass measurements. The quantitative evaluation of parts a) and b) can be based on blade localization accuracy and repeatability, while part c) and part d) can assess aero-engine blade weighing precision. The validation metrics can define the experimental validation protocol.

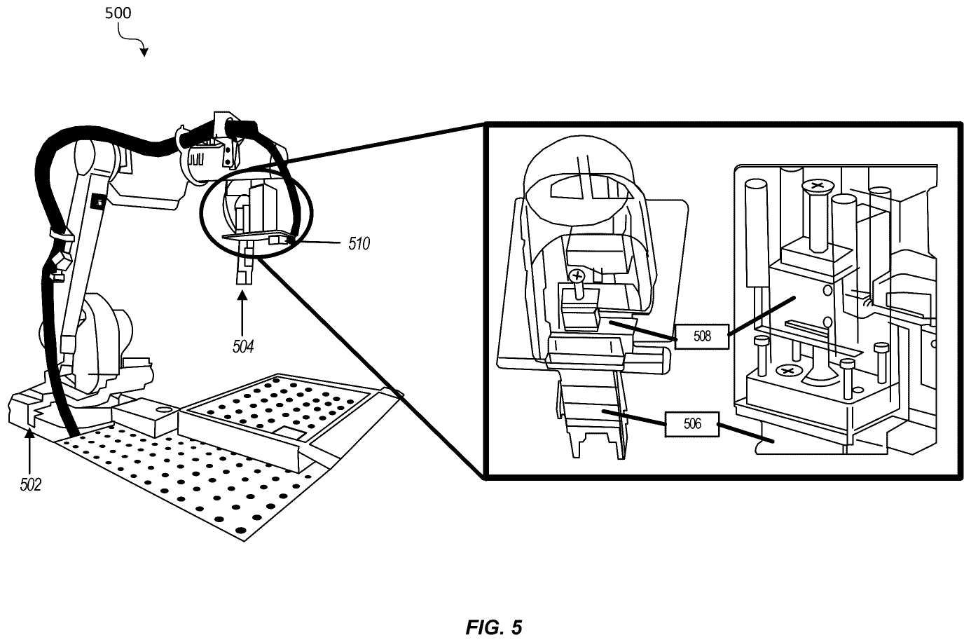

An example of a setup of the aero-engine blade weighing system 500 is illustrated in FIG. 5 in which the system setup can mimic industrial settings and can seamlessly replace operators without restructuring an industrial environment. The system can include a robot manipulator 502 that maneuvers the end effector precisely to the target grasping poses and an end effector 504 that can include any suitable gripper 506 to grasp the engine blades during the weighing process. Additionally, or alternatively, a load cell 508 can be attached in line with the gripper 506 for blade mass measurement. Any suitable numbers (e.g., 22 or others) of geometrically identical blades can be randomly placed in any suitable number (e.g. 60) of equally spaced slots in-plane with an ArUco fiducial marker. The blades may vary in mass, such as ranging from approximately 16 g to approximately 17.3 g, though other masses are possible. The blades and the fiducial marker can be observed by the detector such as the Intel RealSense D405 depth camera for blade detection and localization.

FIG. 5 is a schematic and autonomous robotic system with a camera system and end effector. In some examples, FIG. 5 illustrates an overview of the robotic aero-engine weighing and sorting system setup. The end effector 504 can include a depth camera 510 for blade detection and localization, observing the blade tray. Following detection, the robotic arm moves the end effector 504, enclosing the load cell 508 to inspect and weigh the engine blades.

The system can have numerous advantages over other systems and techniques. It can accelerate the blade weighing process, making it up to or more than nine times faster, thus increasing production throughput. The system achieves exceptional precision, with an aggregate precision and accuracy of 0.0404 and 0.0252 respectively, meeting the stringent requirements of the aerospace industry. Additionally, the system can integrate seamlessly into existing industrial environments without necessitating extensive and costly modifications. Table 3 presents the mass measurements for eight blades along with the mass measurement precision σₘ. In addition to the weighing precision, the weighing accuracy can be evaluated as the mean absolute error (MAE), ηₘ, compared against the blade mass ground truth obtained from a calibrated PS 6100.X2.M mass balance. A constant bias can be obtained for the load cell 508 measurements, which is approximately 0.0.23 g. The bias can be added to the blade mass; consequently, those values are compared to the ground truth of each blade. The error of the proposed system falls between less than 0.0074 grams to less than 0.122 grams.

TABLE 3
illustrates quantified mass measurement precision and accuracy for the
system across eight trials for eight blades in which the obtained mass measurement
aggregate precision σₘ and accuracy ηₘ of the proposed system is
approximately 0.0395 g and approximately 0.0437 g, respectively.

| | Blade | Blade | Blade | Blade | Blade | Blade | Blade | Blade |
|---|---|---|---|---|---|---|---|---|
| Trial ID | ID 1 | ID 2 | ID 3 | ID 4 | ID 5 | ID 6 | ID 7 | ID 8 |
| 1 | 16.6 | 17.09 | 17.05 | 16.71 | 17.39 | 16.46 | 17.1 | 16.53 |
| 2 | 16.61 | 17.08 | 17.1 | 16.71 | 17.41 | 16.49 | 17.24 | 16.43 |
| 3 | 16.57 | 17.08 | 17.07 | 16.61 | 17.43 | 16.49 | 17.12 | 16.49 |
| 4 | 16.68 | 17.05 | 17.09 | 16.7 | 17.41 | 16.48 | 17.15 | 16.54 |
| 5 | 16.64 | 16.96 | 17.08 | 16.68 | 17.42 | 16.51 | 17.2 | 16.54 |
| 6 | 16.66 | 17.02 | 17.07 | 16.69 | 17.39 | 16.54 | 17.21 | 16.5 |
| 7 | 16.64 | 17.05 | 17.08 | 16.64 | 17.42 | 16.55 | 17.15 | 16.53 |
| 8 | 16.63 | 17.15 | 17.1 | 16.7 | 17.34 | 16.49 | 17.28 | 16.45 |
| σ (g) | 0.035 | 0.054 | 0.016 | 0.036 | 0.03 | 0.03 | 0.061 | 0.042 |
| Ground | 16.63 | 17.06 | 17.08 | 16.68 | 17.4 | 16.5 | 17.18 | 16.5 |
| Truth (g) | | | | | | | | |
| η (g) | 0.04 | 0.015 | 0.0128 | 0.05 | 0.043 | 0.003 | 0.036 | 0.002 |
| | | | | | | | σm (g) | 0.0404 |
| | | | | | | | ηm (g) | 0.0252 |

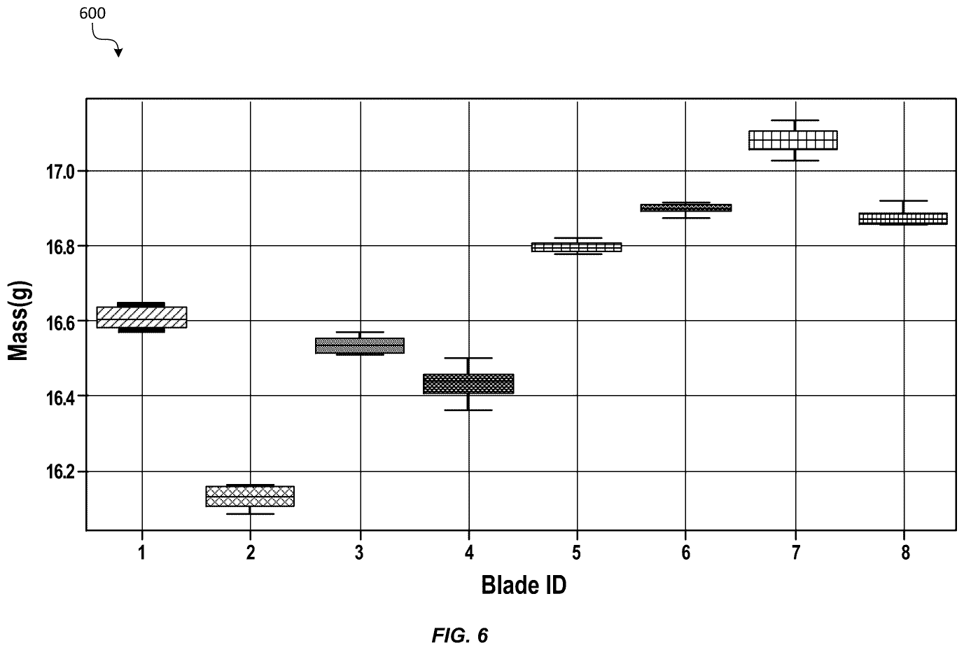

FIG. 6 illustrates a multi-experiment plot 600 evaluating the repeatability of the system across multiple trials. The obtained results demonstrate that the system meets aerospace standards. The proposed robotic perception capabilities can enable seamless adaptation to diverse facilities. The automation of aero-engine blade weighing and sorting can accelerate the task, such as by a factor of nine times or more, and can mitigate the drawbacks to address concerns related to operator error and expediting the blade weighing process.

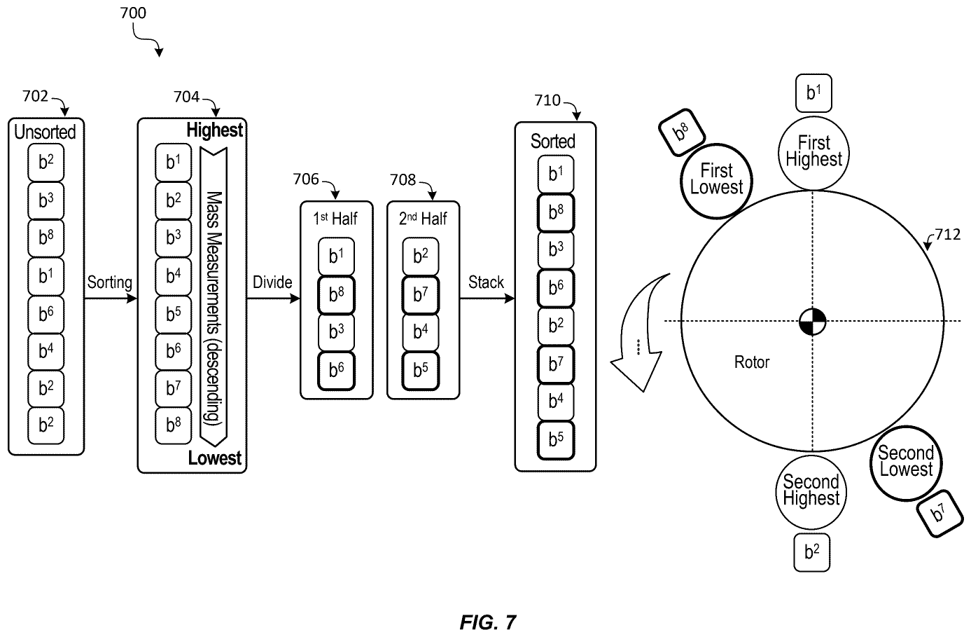

FIG. 7 is a data flow diagram of an example of a process of installing aerospace engine blades into rotors after being sorted in descending mass order 700. Once the robot localizes the target blade, the process proceeds with the robot grasping the blade and measuring its mass. The robot can move the gripper 102 of FIG. 1 to the proximity of the blade and can record an initial load-free measurement from the load cell

$${M}_{initial}^{j} .$$

This measurement can be used to offset the measured blade mass to eliminate any biases in the load cell. The robot can grasp the blade and obtain another measurement from the load cell denoted by

$${M}_{final}^{j} .$$

The grasped blade can be aligned with the gripper of FIG. 1 in the z-axis and the load cell's measuring axis to minimize any moments from the weight of the blade. To account for noise, the measurements can be passed through a low pass filter that averages multiple measurements as

$${M}_{final} = \sum {jM}_{final}^{j} / N , j ϵ [ 1 , N ]$$

in which N denotes the total number of measurements. The mass of the target blade can be calculated as M_{blade}=M_{final}−Mᵢₙᵢₜᵢₐₗ.

The above-described process can be then repeated N times across the whole set of detected blades within the tray of unsorted blades 702. The subsequent sorting procedure entails organizing the blades based on their mass measurements using a specialized technique that ensures the dynamic balance of the aeroengine once the blades are installed. For example, the blades can be arranged in descending arrangement of blade mass 704. The detected blades are then divided into two groups: S1 706 and S2 708. S1 706 initially includes the blades with the highest and lowest masses, while S2 708 contains the second highest and second lowest. Sequentially, S1 706 continues to add blades, ranked third highest and third lowest, while S2 708 accumulates blades in a similar manner. This alternating pattern continues until all blades are sorted. Following this, the S1 706 is stacked on top of S2 708, resulting in a balanced sorting order 710, as illustrated in FIG. 7. The resulting balanced sorting order 710 can then be applied to a rotor 712. The above-described techniques can adhere to the requirements of jet engine rotors, ensuring a well-balanced distribution of measured blades.

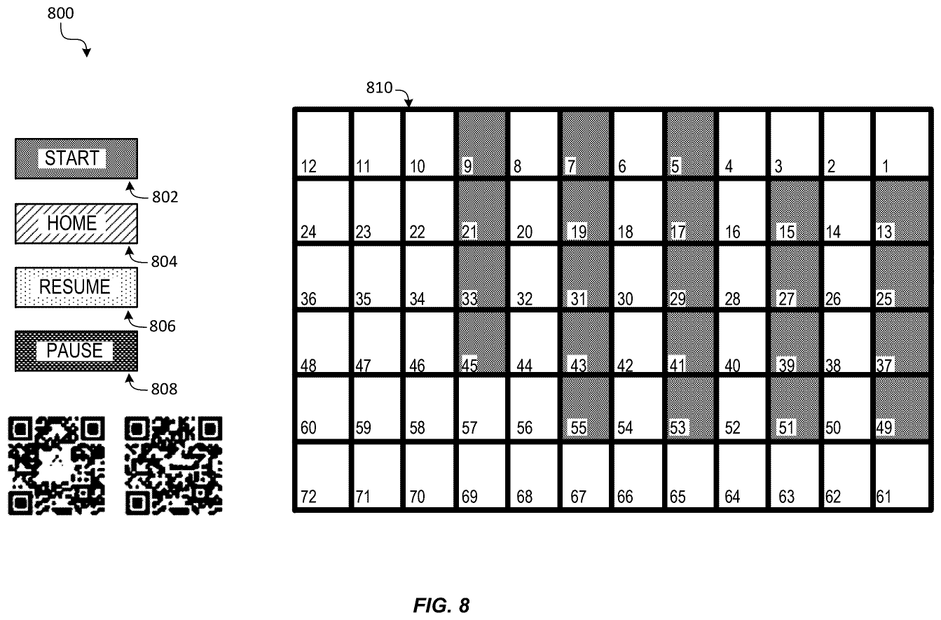

Once the robot estimates the mass of a blade, it reports the measurement to the graphical user interface (GUI) 800 in real-time, as illustrated in FIG. 8. The GUI may include selectable instructions such as a start 802 function, a home 804 function, a resume 806 function or a pause 808 function. The GUI may also show a representation of the blade distribution 810.

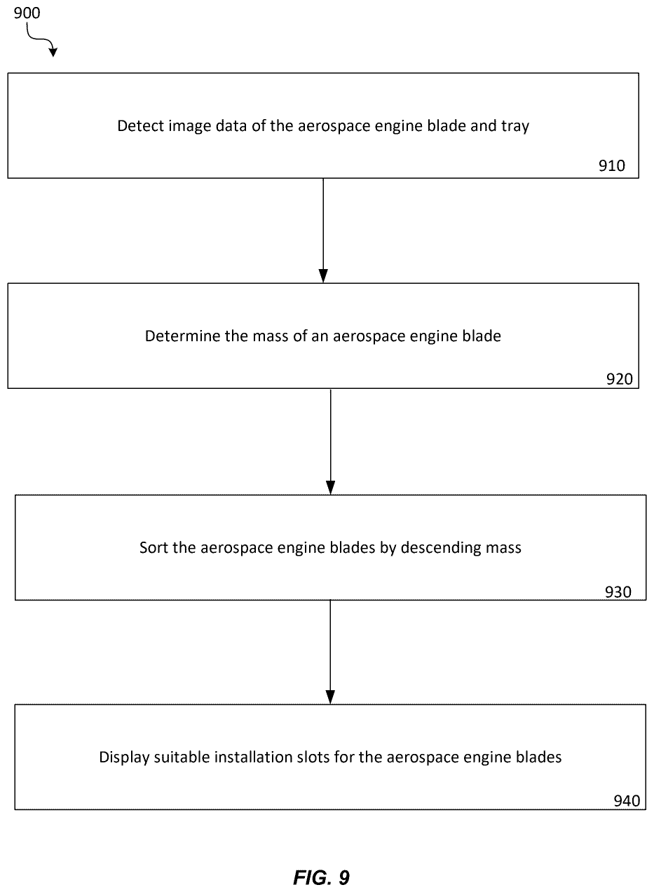

FIG. 9 is a flow chart of an example of an autonomous robotic system measuring and sorting aerospace engine blades 900. At block 910 the robotic system positions itself above the aerospace engine blades and using the camera system 110 of FIG. 1 may detect data using an AI output of the camera system. Another method to detect aerospace engine blade information may be to use fiducial markers 112.

At block 920 the mass of the aerospace engine blade is captured. The robotic system may identify the aerospace engine blade using the camera system 110 of FIG. 1. The robotic arm 108 may position itself above the aerospace engine blade in a tray and use the end effector 106 with a gripper 212 of FIG. 1 and FIG. 2 to grasp the blade. The S-beam load cell 204 may then determine the weight of the aerospace engine blade.

At block 930 an aerospace engine blade is sorted. After a weight is acquired an AI model can sort the blades such as in descending order or other suitable orders. The blades may be placed into two groups and the first highest and lowest aerospace engine blade weight in a set is placed in the first group and the second highest and lowest mass may be placed in the second group. This pattern maybe repeated with the next set of aerospace engine blades being placed in the first group and continued until all the blades are sorted.

At block 940 the suitable installation slots are displayed. The first and second set are placed on top of each other. This is then displayed on a command computer 214 of FIG. 2. The display may be shown as the GUI 800 of FIG. 8. In some embodiments the displayed information may direct a user to install the aerospace engine blades in a most suitable installation pattern on the aerospace engine.

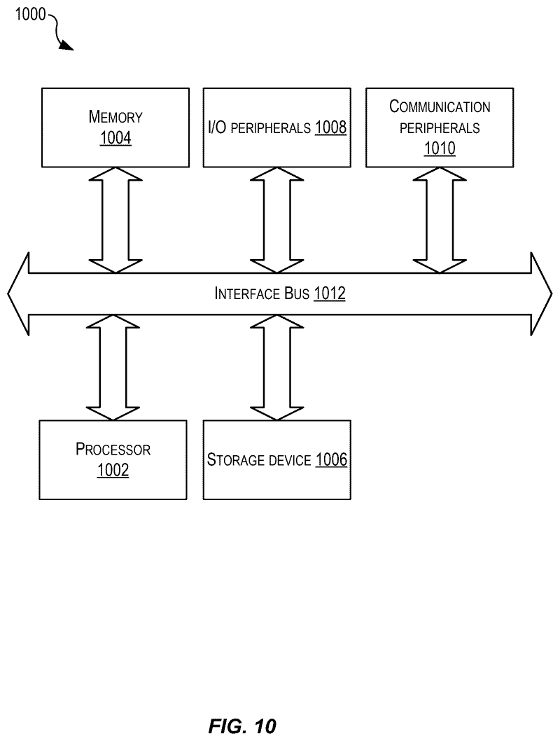

FIG. 10 illustrates examples of components of a computer system 1000, according to at least one example. The computer system 1000 may be a single computer such as a user computing device and/or can represent a distributed computing system such as one or more server computing devices. In some examples, the computer system 1000 may be configured to control the operation of one or more automated elements of the autonomous robotic system 100 of FIG. 1, and any other automated equipment, of a warehouse, manufacturing facility, bottling facility, packing facility, or the like.

The computer system 1000 may include at least a processor 1002, a memory 1004, a storage device 1006, input/output peripherals 1008, communication peripherals 1010, and an interface bus 1012. The interface bus 1012 is configured to communicate, transmit, and transfer data, controls, and commands among the various components of the computer system 1000. The memory 1004 and the storage device 1006 include computer-readable storage media, such as Radom Access Memory (RAM), Read ROM, electrically erasable programmable read-only memory (EEPROM), hard drives, CD-ROMs, optical storage devices, magnetic storage devices, electronic non-volatile computer storage, for example Flash® memory, and other tangible storage media. Any such computer-readable storage media can be configured to store instructions or program codes embodying aspects of the disclosure. The memory 1004 and the storage device 1006 also include computer-readable signal media. A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with the computer system 1000.

Further, the memory 1004 includes an operating system, programs, applications, and/or other software, models, modules, and the like. The processor 1002 is configured to execute the stored instructions and includes, for example, a logical processing unit, a microprocessor, a digital signal processor, and other processors. The memory 1004 and/or the processor 1002 can be virtualized and can be hosted within another computing system of, for example, a cloud network or a data center. The input/output peripherals 1008 include user interfaces, such as a keyboard, screen (e.g., a touch screen), microphone, speaker, other input/output devices, and computing components, such as graphical processing units, serial ports, parallel ports, universal serial buses, and other input/output peripherals. The input/output peripherals 1008 are connected to the processor 1002 through any of the ports coupled to the interface bus 1012. The communication peripherals 1010 are configured to facilitate communication between the computer system 1000 and other computing devices over a communications network and include, for example, a network interface controller, modem, wireless and wired interface cards, antenna, and other communication peripherals.

What is claimed is:

1. A system comprising:
a robotic manipulator;
an end effector with a gripping mechanism coupled with the robotic manipulator to facilitate manipulation of one or more aerospace engine blades of an aerospace engine;
a measurement unit coupled with the robotic manipulator to perform mass and weight measurements of the one or more aerospace engine blades as the one or more aerospace engine blades are gripped by the robotic manipulator;
an imaging sensor communicatively coupled with the robotic manipulator, the end effector, and/or an imaging system for capturing image data about the one or more aerospace engine blades; and
a computing system configured to perform operations comprising:
detecting the image data about the one or more aerospace engine blades using a neural network;
refining the detected image data for detecting the one or more aerospace engine blades;
causing the robotic manipulator, using the end effector, to grasp and measure an aerospace engine blade of the one or more aerospace engine blades such that a mass measurement of the aerospace engine blade is generated by the measurement unit;
sorting mass of the aerospace engine blade using the neural network by comparing the mass measurement to masses of other aerospace engine blades of the one or more aerospace engine blades being measured;
sorting, based on output from the neural network, the one or more aerospace engine blades based on the mass measurement and according to a dynamic balancing setting associated with the aerospace engine; and
outputting a command comprising instructions executable to display the data throughout a process including an optimized installation slot in the aerospace engine for each of the aerospace engine blades to ensure dynamic balancing.

2. The system of claim 1, wherein the computing system is coupled with a separate dynamic balancing system to perform dynamic balancing of the aerospace engine by rotating the aerospace engine at one or more predetermined speeds for evaluating imbalance, and wherein the computing system is configured to receive imbalance data from the separate dynamic balancing system and refine a sorting order and a slot allocation in the aerospace engine for each blade to ensure optimal balance and performance.

3. The system of claim 1, wherein the computing system is further configured to:
utilize the image data to store a pixel data of a location of the aerospace engine blade within a tray; and
center the aerospace engine blade in an image plane according to the pixel data for detection of the aerospace engine blade.

4. The system of claim 1, wherein the computing system is further configured to:
record a first measurement of a load free load cell;
grasp the aerospace engine blade; and
record a second measurement of a load cell with the aerospace engine blade grasped by the load cell.

5. The system of claim 4, wherein the computing system is further configured to process of the mass of an aerospace engine blade by computing
$${M}_{final} = \sum {jM}_{final}^{j} / N , j ϵ [ 1 , N ] ,$$
wherein M_{final} is the mass of the aerospace engine blade, M^{j} is the mass of the second measurement, and N is the total number of measurements.

6. The system of claim 4, wherein the computing system is further configured to perform process a final mass of the aerospace engine blade by M_{blade}=M_{final}−Mᵢₙᵢₜᵢₐₗ wherein M_{blade} is the final mass of the aerospace engine blade, and Mᵢₙᵢₜᵢₐₗ is the first measurement.

7. The system of claim 1, wherein the computing system is further configured to:
sort a set of aerospace engine blades into two groups based on a final mass of the aerospace engine blade within a first group is and a second group;
organize the aerospace engine blade in descending order through a process comprising:
sorting a highest mass and a lowest mass into the first group;
sorting a second highest mass and a second lowest mass into the second group; and
sorting a next highest mass and a next lowest mass alternating between the first group and the second group;
continue sorting until all the aerospace engine blades are sorted; and
control the robotic manipulator to stack the first group on top of the second group.

8. A method comprising:
detecting a mass of a first object of a plurality of objects as the object is gripped by a robotic manipulator;
capturing image data about the plurality of objects using an imaging sensor communicatively coupled with an artificial intelligence (AI) model of the robotic manipulator, an end effector, and/or an imaging system;
causing the robotic manipulator, using the end effector, to grasp a second object of the one or more objects such that a mass of the second object is determined, the second object being the same as, or different from, the first object; and
sorting the plurality of objects based on the mass according to a dynamic balancing parameter of an end system for the plurality of objects, the dynamic balancing parameter resulting in a physical distribution of the plurality of objects.

9. The method of claim 8, further comprising:
capturing and storing images of the second object; and
processing the images to create a bounding box around the second object utilizing the AI model.

10. The method of claim 8, further comprising;
recording a first measurement of a load-free load cell;
grasping the second object; and
recording a second measurement of a load cell with the second object grasped by the load cell.

11. The method of claim 10, further comprising calculating the mass of the second object by computing
$${M}_{final} = \sum {jM}_{final}^{j} / N , j ϵ [ 1 , N ] ,$$
wherein M_{final} is the mass of the second object, M^{j} is the mass of the second measurement, and N is the total number of measurements.

12. The method of claim 10, further comprising processing a final mass of the second object by M_{blade}=M_{final}−Mᵢₙᵢₜᵢₐₗ wherein M_{blade} is the final mass of the second object, and Mᵢₙᵢₜᵢₐₗ is the first measurement.

13. The method of claim 8, further comprising:
sorting a subset of the plurality of objects into two groups based on a final mass of the subset of the plurality of objects within a first group and a second group; and
organizing the subset in descending order by:
sorting a highest mass and a lowest mass into the first group;
sorting a second highest mass and a second lowest mass into the second group;
sorting a next highest mass and a next lowest mass alternating between the first group and the second group; and
continuing to sort until all objects in the subset are sorted.

14. The method of claim 13, further comprising controlling the robotic manipulator to stack the first group on top of the second group and display an output including weights of objects included in the subset on a graphical user interface (GUI).

15. A non-transitory computer-readable medium comprising instructions that are executable by a processing device for causing the processing device to perform operations comprising:
detecting a mass of a first object of a plurality of objects as the object is gripped by a robotic manipulator;
capturing image data about the plurality of objects using an imaging sensor communicatively coupled with an artificial intelligence (AI) model of the robotic manipulator, an end effector, and/or an imaging system;
causing the robotic manipulator, using the end effector, to grasp a second object of the one or more objects such that a mass of the second object is determined, the second object being the same as, or different from, the first object; and
sorting the plurality of objects based on the mass according to a dynamic balancing parameter of an end system for the plurality of objects, the dynamic balancing parameter resulting in a physical distribution of the plurality of objects.

16. The non-transitory computer-readable medium of claim 15, further comprising instructions that are executable by the processing device for causing the processing device to perform further operations comprising:
capturing and storing images of the second object; and
processing the images to create a bounding box around the second object utilizing the AI model.

17. The non-transitory computer-readable medium of claim 15, further comprising instructions that are executable by the processing device for causing the processing device to perform further operations comprising:
recording a first measurement of a load-free load cell;
grasping the second object; and
recording a second measurement of a load cell with the second object grasped by the load cell.

18. The non-transitory computer-readable medium of claim 17, further comprising instructions that are executable by the processing device for causing the processing device to perform further operations comprising calculating the mass of the second object by computing
$${M}_{final} = \sum {jM}_{final}^{j} / N , j ϵ [ 1 , N ] ,$$
wherein M_{final} is the mass of the second object, M^{j} is the mass of the second measurement, and N is the total number of measurements.

19. The non-transitory computer-readable medium of claim 17, further comprising instructions that are executable by the processing device for causing the processing device to perform further operations comprising processing a final mass of the second object by M_{blade}=M_{final}−Mᵢₙᵢₜᵢₐₗ, wherein M_{blade} is the final mass of the second object, and Mᵢₙᵢₜᵢₐₗ is the first measurement.

20. The non-transitory computer-readable medium of claim 15, further comprising instructions that are executable by the processing device for causing the processing device to:
sort a subset of the plurality of objects into two groups based on a final mass of the subset of the plurality of objects within a first group and a second group; and
organize the subset in descending order by:
sorting a highest mass and a lowest mass into the first group;
sorting a second highest mass and a second lowest mass into the second group;
sorting a next highest mass and a next lowest mass alternating between the first group and the second group; and
continuing to sort until all objects in the subset are sorted.
